(19) United States
(12) Patent Application Publication
(10) Pub. No.: US 2011/0168186 A1
(43) Pub. Date: Jul. 14, 2011

(54) INTRA-ORAL APPLIANCE FOR TREATMENT OF SLEEP DISORDERS

(76) Inventor: Leonard Wayne HALSTROM, Lion's Bay (CA)

(21) Appl. No.: 12/687,062

(22) Filed: Jan. 13, 2010

(51) Int. Cl. A61F 5/56; A61C 5/14

(52) U.S. Cl. 128/848; 128/861

(57) ABSTRACT

An intra-oral dental appliance is provided for treatment of sleep disorders including snoring, sleep apnea and nocturnal bruxism. The appliance is particularly suited for use by patients having class II malocclusion and includes an upper member conforming to the patient's maxillary dentition; a lower member conforming to the patient's mandibular dentition; and a connecting assembly for adjustably coupling the upper and lower members together. The connecting assembly includes a first element mounted on the upper member, the first element having a cavity therein; a second element mounted on the lower member, the second element comprising a posterior portion and an adjustment bar extending anteriorly from the posterior portion and having a plurality of spaced-apart adjustment positions formed therein; and a connector having a first end loosely captured within the cavity and a second end adjustably connectable to a selected one of the adjustment positions located on the adjustment bar. The connecting assembly maintains the lower member in a protruded position relative to its normal position to maintain the patency of the user's pharnygeal airway while permitting a limited degree of movement of the lower member relative to the upper member in the protruded position. In use, the adjustment bar extends substantially anteriorly of the patient's mandibular dentition to avoid constraining the patient's tongue, thereby enhancing the comfort and wearability of the appliance.

### TECHNICAL FIELD

This application relates to an intra-oral appliance for treatment of sleep disorders.

### BACKGROUND

Several medical disorders appear to be related to partial or complete obstruction of an individual's pharyngeal airway during sleep. As described in the applicant's prior U.S. Pat. Nos. 5,365,945, 6,041,784, 6,161,542 and 6,729,335, the disclosures of which are hereby incorporated by reference, snoring and obstructive sleep apnea are typically caused by complete or partial obstruction of an individual's pharyngeal airway resulting from the apposition of the rear portion of the tongue or soft palate with the posterior pharyngeal wall. Obstructive sleep apnea is a potentially lethal disorder in which breathing stops during sleep for 10 seconds or more, sometimes up to 300 times per night. Snoring occurs when the pharyngeal airway is partially obstructed, resulting in vibration of the oral tissues during respiration. These sleep disorders tend to become more severe as patients grow older, likely due to a progressive loss of muscle tone in the patient's throat and oral tissues.

Habitual snoring and sleep apnea have been associated with other potentially serious medical conditions, such as hypertension, ischemic heart disease and strokes. Accordingly, early diagnosis and treatment is recommended. One surgical approach, known as uvulopalatopharyngoplasty, involves removal of a portion of the soft palate to prevent closure of the pharyngeal airway during sleep. However, this operation is not always effective and may result in undesirable complications, such as nasal regurgitation.

A wide variety of non-surgical approaches for treating sleep disorders have also been proposed including the use of oral cavity appliances. It has been previously recognized that movement of the mandible (lower jaw) forward relative to the maxilla (upper jaw) can eliminate or reduce sleep apnea and snoring symptoms by causing the pharyngeal air passage to remain open. Several intra-oral dental appliances have been developed which the user wears at night to maintain the mandible in an anterior, protruded (i.e. forward) position. Such dental appliances essentially consist of acrylic or elastomeric bite blocks, similar to orthodontic retainers or athletic mouth guards, which are custom-fitted to the user's upper and lower teeth and which may be adjusted to vary the degree of anterior protrusion.

One shortcoming of some prior art intra-oral cavity appliances is that they take up a significant amount of room within a patient's mouth which reduces the space remaining to accommodate the patient's tongue. This is of particular concern in patient's suffering from class II malocclusion, i.e. an “underbite” condition where the patient's mandible or lower jaw is displaced significantly posteriorly of the upper jaw. Prior art intraoral appliances may significantly constrain the patient's tongue in such patients and be uncomfortable to wear, resulting in reduced patient compliance to dental appliance therapy.

The need has therefore arisen for an intra-oral appliance suitable for treatment of sleep disorders which is adapted for use by patients having class II malocclusion.

### BRIEF DESCRIPTION OF DRAWINGS

In drawings which describe embodiments of the invention but which should not be construed as restricting the spirit or scope thereof,

FIG. 1 is a schematic, side elevational view of the upper pharyngeal region of a patient suffering from obstructive sleep apnea or snoring and having a normal bite profile.

FIG. 2 is a schematic, side elevational view of the upper pharyngeal region of a patient suffering from obstructive sleep apnea or snoring and having class II malocclusion.

FIG. 3 is a schematic, side elevational view of the upper pharyngeal region of the patient of FIG. 2 fitted with a prior art dental appliance.

FIG. 4 is a schematic, side elevational view of the upper pharyngeal region of the patient of FIG. 2 fitted with a dental appliance configured in accordance with an embodiment of the invention.

FIG. 5 is a schematic, side elevational view of the dental appliance of FIG. 4 at an alternative adjustment setting.

FIG. 6 is a schematic, side elevational view of the dental appliance of FIG. 4 at a further alternative adjustment setting and showing an anterior portion of the adjustment bar truncated.

FIG. 7 is an enlarged, exploded view of the connecting assembly of the applicant's dental appliance for connecting upper and lower bite blocks shown in dotted outline.

FIG. 8 is an enlarged, longitudinal sectional view of the dental appliance of FIG. 4 showing the stylus connector inserted in a forwardmost threaded aperture of the adjustment bar.

FIG. 9 is an enlarged, longitudinal sectional view of the dental appliance of FIG. 5 showing the stylus connector inserted in a central threaded aperture of the adjustment bar.

FIG. 10 is an enlarged, longitudinal sectional view of the dental appliance of FIG. 6 showing the stylus connector inserted in a rearmost threaded aperture of the adjustment bar and showing an anterior portion of the adjustment bar truncated.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

FIG. 1 is a schematic, side elevational view of the upper pharyngeal region of a typical patient suffering from obstructive sleep apnea or snoring and having a normal bite profile. When the patient is asleep the oral cavity tissues relax and the distal (rear) portion of the tongue 10 tends to slide rearwardly toward the posterior pharyngeal wall 12. This may result in partial or complete occlusion of the pharyngeal airway 14. As the volume of airway 14 diminishes, the velocity of the air passing the oral cavity tissues tends to increase. This may result in vibration of the oral cavity tissues, such as the uvula 16 which is a fleshy projection suspended from the soft palate 18 over the root of tongue 10. The vibration of these oral tissues causes the snoring sound.

The patient of FIG. 1 has a normal bite profile. That is, the mandible or lower jaw 20 is aligned slightly posteriorly of the upper jaw 22 so that the lower dentition 24 and upper dentition 26 engage one another. The patient's lower lip 28 is also generally aligned with the upper lip 30. In normal occlusion the teeth meet with an overjet of approximately 2 mm (i.e. the extent to which the lower incisor teeth are positioned posterior to the upper incisor teeth) and an overbite of approximately 2 mm (i.e. the extent to which the tip of the lower incisor teeth are positioned posterior to the upper incisor teeth).

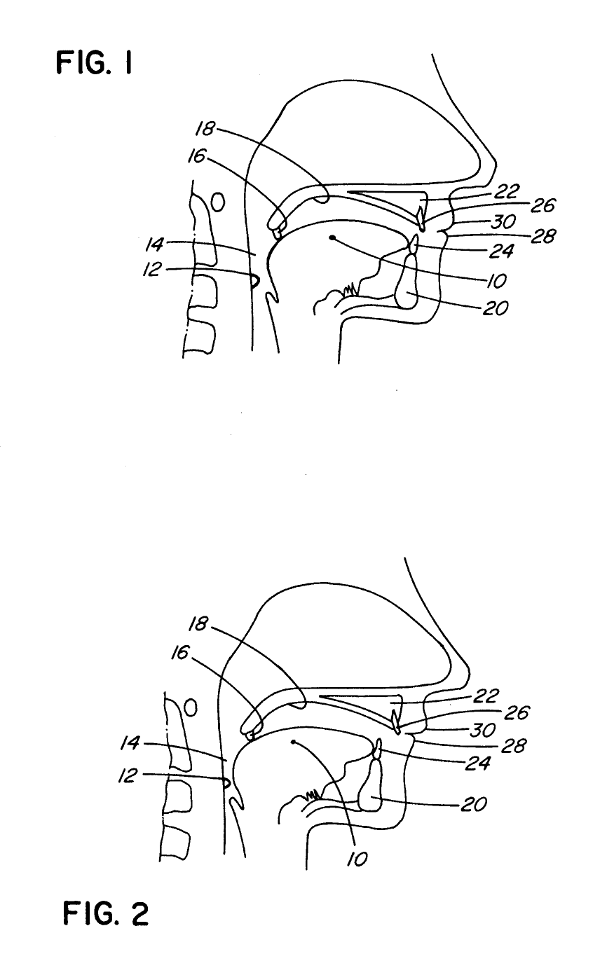

FIG. 2 illustrates the anatomy of a patient having class II malocclusion. In this case the patient's jaw 20 is displaced significantly posteriorly of the upper jaw 22. The lower lip 28 is also displaced posteriorly of the upper lip 30. Malocclusions are generally classified for dental purposes as class II when the overjet exceeds about 2 mm. In patients having class II malocclusion, the distal portion of tongue 10 may be proportionately closer to pharyngeal wall 12 thereby reducing the size of the pharyngeal airway 14. This may in turn exacerbate snoring and sleep apnea symptoms.

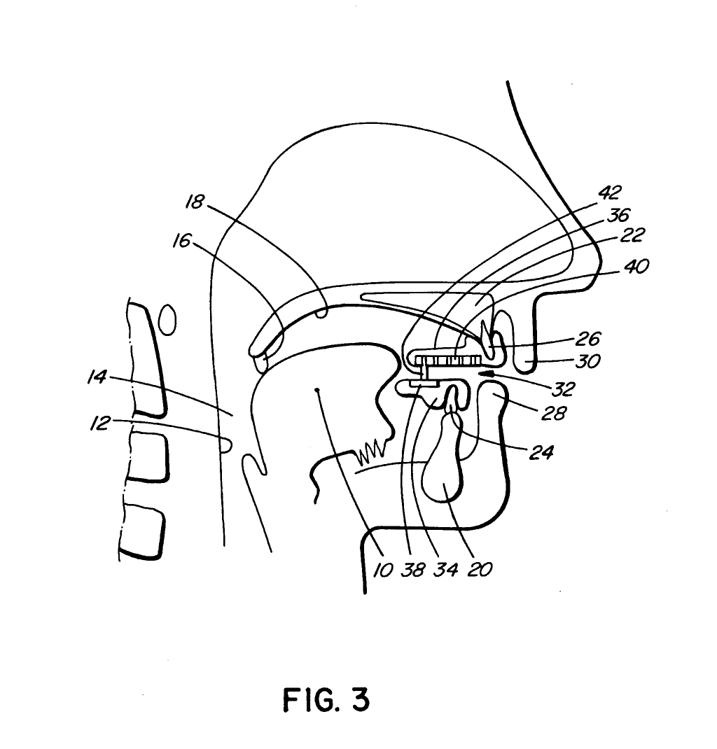

Patients having class II malocclusion may encounter difficulties wearing prior art dental appliances, such as the appliance described in U.S. Pat. No. 6,729,335 issued May 4, 2004 and having common inventorship. FIG. 3 illustrates the mouth anatomy of a patient having class II malocclusion fitted with such a prior art dental appliance 32. Appliance 32 causes the individual's mandible 20 to be moved to an anterior, protruded position relative to its normal, unadjusted position. This causes the dorsal surface of the tongue 10 to move forwardly away from the posterior pharyngeal wall 12 which in turn results in an increase in volume of the pharyngeal airway 14 as described above. It has been shown that maintaining airway 14 substantially open during sleep alleviates the undesirable symptoms associated with obstructive sleep apnea and snoring.

The extent of anterior protrusion of prior art appliance 32 may be adjusted by altering the relative position of a lower bite block 34 relative to an upper bite block 36 (FIG. 3). In particular, the position of a lower element 38 relative to an upper element 40 can be adjusted by means of a connector 42.

As shown in FIG. 3, a substantial portion of lower bite block 34 extends posteriorly of lower dentition 24. In patients with class II malocclusion, this may constrain the space available for tongue 10 within the oral cavity. The constrained position of the tongue may be uncomfortable and result in non-compliance with dental appliance therapy and hence a re-emergence of snoring and sleep apnea symptoms.

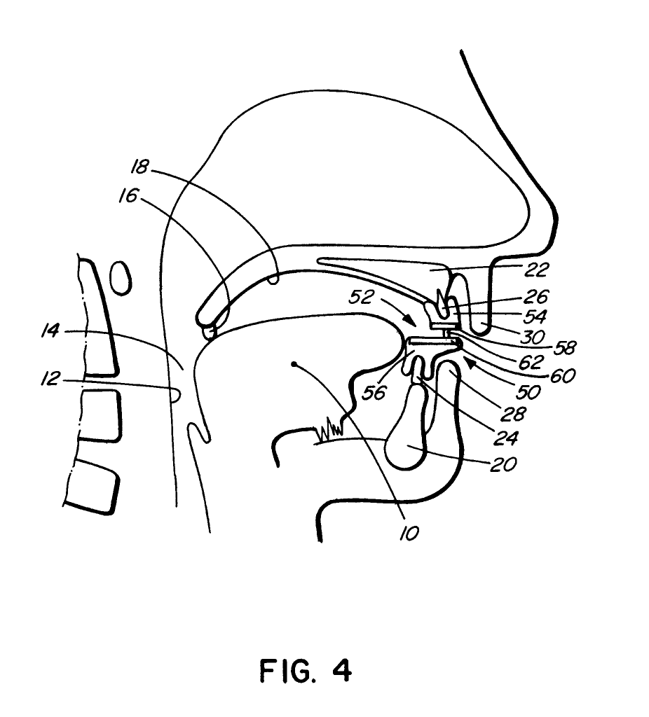

FIG. 4 shows a patient with class II malocclusion fitted with a dental appliance configured in accordance with an embodiment of the present invention. Appliance 50 includes a connecting assembly 52 for adjustably coupling an upper bite block or member 54 and a lower bite block or member 56 together. Bite blocks 54, 56 are formed from an elastomeric material molded to conform to the patient's dentition. As explained above, connecting assembly 52 is configured so that lower bite block 56, and hence the patient's lower jaw 20, is maintained in an anteriorly protruded (i.e. forward) position relative to its normal, unadjusted position while still permitting some relative movement of bite blocks 54, 56 in the protruded position.

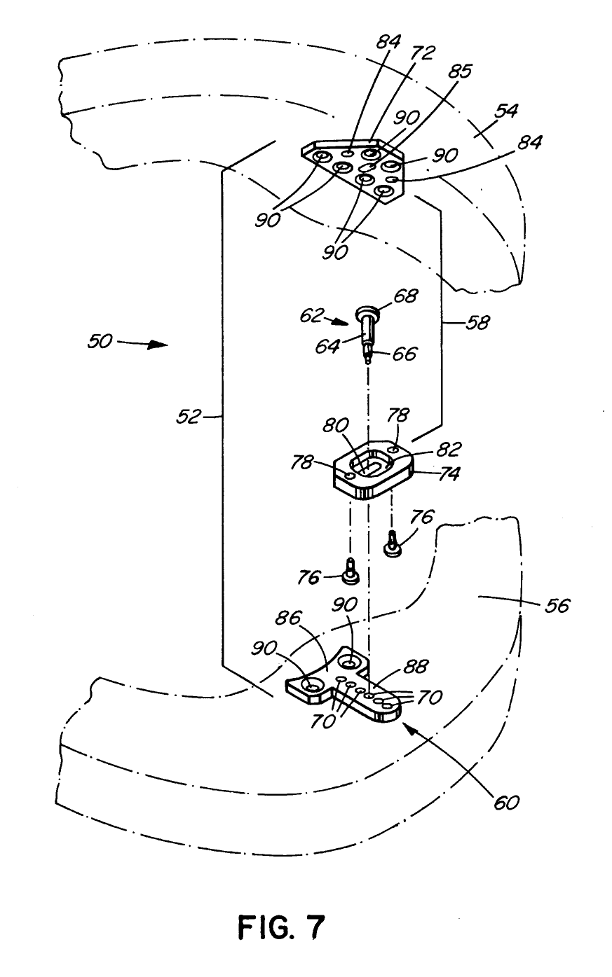

As shown best in FIG. 7, connecting assembly 52 includes a first connecting element 58 secured to upper bite block 54 and a second connecting element 60 secured to lower bite block 56. Connecting elements 58, 60 are adjustably coupled together by a stylus connector 62 having a shaft 64 and a threaded portion 66 at one end thereof and an enlarged head 68 at another end thereof.

As shown in the drawings, stylus shaft 64 extends downwardly and threaded portion 66 may be threadedly coupled to a selected one of a series of internally threaded apertures 70 spaced apart on second connecting element 60. Stylus head 68 is loosely captured within a cavity 82 formed within connecting element 58. As described further below, stylus 62 is capable of moving to a limited extent relative to first connecting element 58 which enables lower bite block 56 to move relative to upper bite block 54 when appliance 50 is worn by a user.

In the illustrated embodiment, first connecting element 58 comprises a first plate 72 and a second plate 74 which may be releasably coupled together (FIG. 7). For example, screw fasteners 76 may be passed through apertures 78 formed in second plate 74 and coupled to internally threaded apertures 84 formed in first plate 72. As will be apparent to a person skilled in the art, many other means for fastening plates 72, 74 together may be envisaged. For example, apertures 78 may also be internally threaded for receiving fasteners 76. Second plate 74 includes an aperture 80 for allowing stylus shaft 64 to pass therethrough As best shown in FIGS. 7-10, when connecting plates 72 and 74 are coupled together they together define cavity 82 for loosely capturing the head 68 of stylus 62. First plate 72 includes a central aperture 85 to permit access to stylus head 68. Accordingly, stylus 62 can be manually turned using a screwdriver or some other suitable tool after first and second plates 72, 74 have been coupled together.

As shown best in FIG. 7, second connecting element 60 is generally T-shaped in the illustrated embodiment. More particularly, element 60 may include a posterior portion 86 and an anteriorly extending adjustment bar 88. Posterior portion 86 may include countersunk retention holes 90 to allow soft elastomeric dental acrylic to flow therein to ensure a strong bond between second connecting element 60 and lower bite block 56. As indicated above, apertures 70 for receiving stylus connector threaded portion 66 are located at spaced intervals on adjustment bar 88. Stylus connector 62 may be threadedly coupled to a selected one of apertures 70 as described below.

In use, appliance 50 may be fabricated in manner similar to prior art dental appliances, such as the appliances described in U.S. Pat. Nos. 5,365,945, 6,041,784, 6,161,542 and 6,729,335. Dental appliance 50 is custom-fitted to suit the requirements of each individual patient. Usually the first step in the fitting procedure is for the dentist or physician to assess the natural range of motion of the patient's jaw and the likely degree of pharyngeal occlusion. This may be determined by physical examinations, sleep studies, x-rays and the like.

Molds of the patient's existing mandibular and maxillary dentition 24, 26 are then taken to enable casting of bite blocks 56, 54. As indicated above, bite blocks 54, 56 are preferably formed of a moldable elastomeric material. The casting process results in a trough formed in lower bite block 56 conforming to the patient's mandibular dentition 24 and a trough formed in upper bite block 54 conforming to the patient's maxillary dentition 26. After bite blocks 54, 56 have been fabricated as aforesaid, they are united by means of connector assembly 52 (FIG. 7). First plate 72 of first connecting element 58 is bonded to the undersurface of upper bite block 54 and second connecting element 60 is bonded to the upper surface of lower bite block 56 by means of heat-cureable dental acrylic. The soft acrylic flows through retention apertures 90 formed on connecting elements 58, 60 to ensure that connecting assembly 52 is securely set in place as the acrylic hardens. As explained above, retention apertures 90 are countersunk to increase the surface area available for bonding to the dental acrylic.

Preferably connecting elements 58, 60 are constructed from commercially pure titanium or some other metal which is non-reactive with oral fluids. Stylus connector 62 and screws 76 are preferably fabricated from titanium or stainless steel. Connecting plates 72 and 74 of connecting element 58 are coupled together as described so that the threaded portion 66 of stylus connector 62 protrudes downwardly through the elongated aperture 80 formed in connecting element 58 (FIG. 7).

Dental appliance 50 is particularly suited for use by patients having class II malocclusion. As explained above, prior art dental appliances may constrain the room available in the mouth for tongue 10 (FIG. 3). As shown in FIGS. 4-6, appliance 50 addresses this shortcoming by positioning a substantial portion of appliance 50 forwardly of lower jaw 20, thus allowing more room for tongue 10 within the oral cavity. In one embodiment, a posterior wall of appliance 50 extends less than 5 mm posteriorly of the trough formed in lower bite block 56 receiving the patient's mandibular dentition. The extent of protrusion of lower jaw 20 is adjustable by selectively inserting stylus connector 62 into one of the apertures 70 on adjustment bar 88. Unlike some prior art dental appliances, incremental advancement of lower jaw 20 is achieved by inserting stylus connector 62 into a progressively more rearward aperture 70.

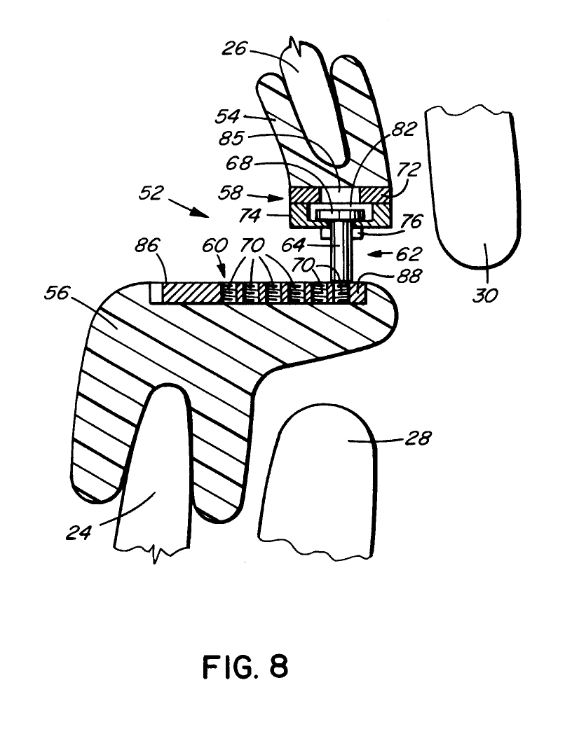

More particularly, FIGS. 4 and 8 show stylus connector 62 threadedly connected to the forwardmost aperture 70 of adjustment bar 88. In this embodiment, all of the apertures 70 of adjustment bar 88 extend forwardly of lower jaw 20 and lower dentition 24 and a portion of lower bite block 56 cantilevers over the patient's lower lip 28. As explained above, since stylus head 68 is loosely captured within cavity 82 (FIG. 8), a limited degree of relative movement of lower bite block 56 relative to upper bite block 54 in a plurality of directions is permitted while maintaining lower jaw 20 in a protruded position to alleviate snoring and sleep apnea symptoms.

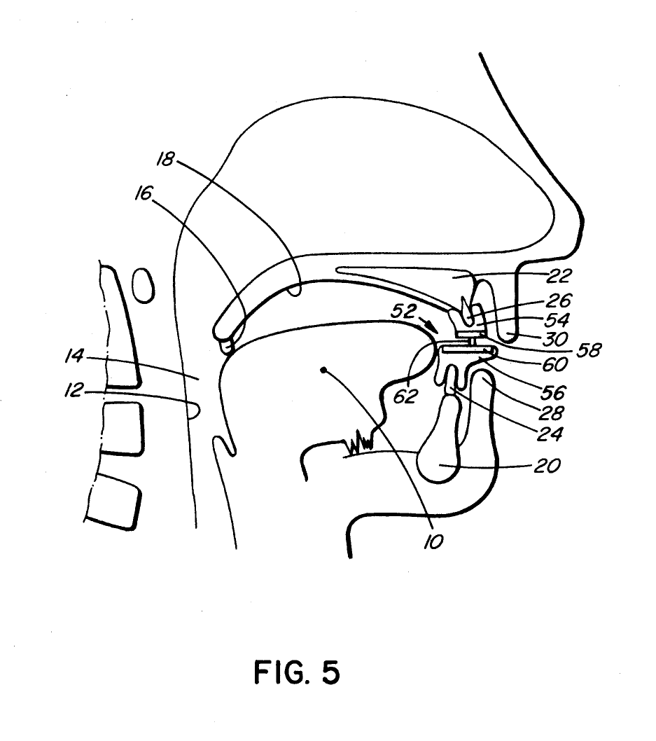

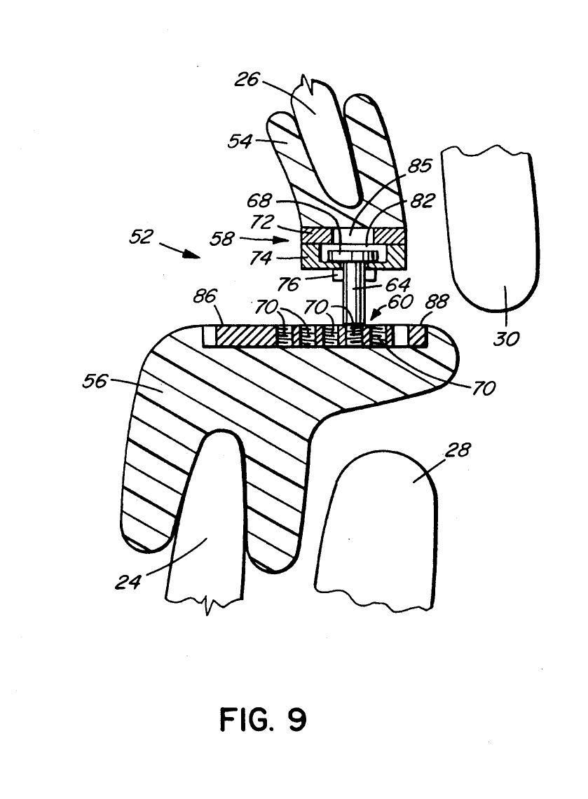

FIGS. 5 and 9 show stylus connector 62 threadedly connected to a central aperture 70 of adjustment bar 88. By inserting stylus connector 62 into a more rearward aperture 70 in comparison to the configuration of FIGS. 4 and 8, the patient's lower jaw 20 is incrementally advanced forwardly. In this configuration, lower bite block 56 continues to cantilever over the patient's lower lip.

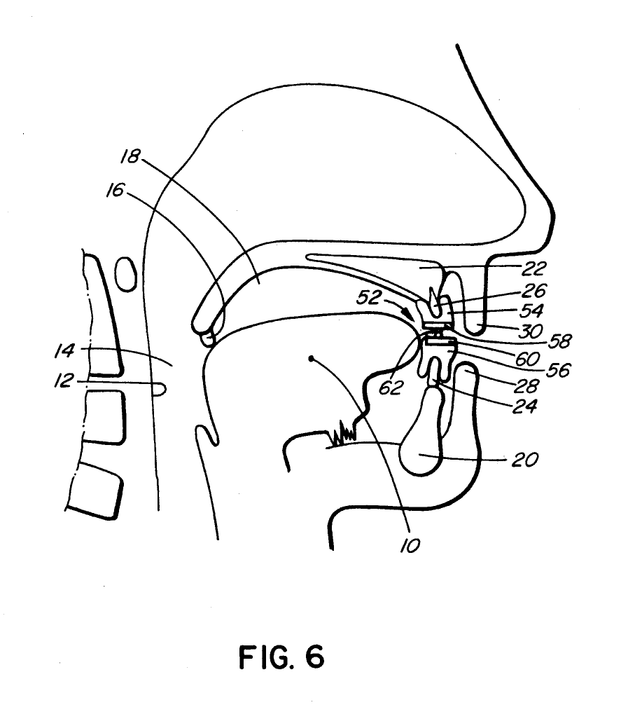

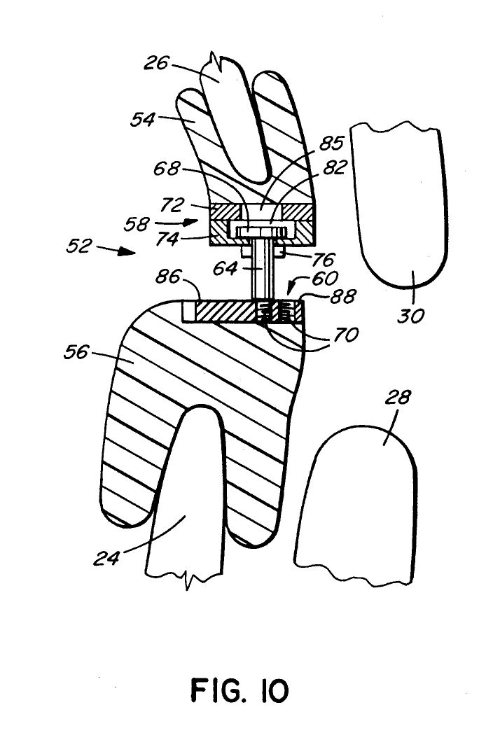

FIGS. 6 and 10 show stylus connector 62 threadedly connected to the rearmost aperture 70 of adjustment bar 88. By inserting stylus connector 62 into a more rearward aperture 70 in comparison to the configuration of FIGS. 5 and 9, the patient's lower jaw 20 is further incrementally advanced forwardly. In this configuration, the forward portion of lower bite block 56, including the forward portion of adjustment bar 88, may be optionally truncated so that bite block 56 no longer cantilevers over the patient's lower lip 28. After truncation, the forward portion of bite block 56 may be remodeled accordingly. As will be appreciated by a person skilled in the art, bite block 56 would not be truncated until after it was determined that one of the rearmost apertures 70 were the optimal settings for the patient. Truncation of bite block 56 may enhance the comfort and wearability of appliance 50 by providing additional room for the patient's lower lip 28.

As will be apparent to those skilled in the art in the light of the foregoing disclosure, many alterations and modifications are possible in the practice of this invention without departing from the spirit or scope thereof. For example, in an alternative embodiment of the invention bite blocks 54, 56 may be cast by the user in a manner similar to readily available over-the-counter sports mouth guards rather than custom-fitted by a dentist, physician or other clinician. Many other variations and alternatives are possible. Accordingly, the scope of the invention is to be construed in accordance with the substance defined by the following claims.

What is claimed is:

1. A dental appliance for wear by a patient having class II malocclusion for treatment of sleep disorders comprising:
(a) an upper member conforming to the patient's maxillary dentition;
(b) a lower member conforming to the patient's mandibular dentition; and
(c) a connecting assembly for coupling said upper and lower members together, wherein said connecting assembly comprises:
(i) a first element mounted on said upper member, said first element having a cavity therein;
(ii) a second element mounted on said lower member, said second element comprising a posterior portion and an adjustment bar extending anteriorly from said posterior portion and having a plurality of spaced-apart adjustment positions formed therein; and
(iii) a connector having a first end captured within said cavity and a second end adjustably connectable to a selected one of said adjustment positions,
wherein said connecting assembly maintains the patient's mandible in an anteriorly protruded position relative to its normal, unadjusted position while permitting a limited degree of movement of said lower member relative to said upper member in said anteriorly protruded position.

2. The dental appliance as defined in claim 1, wherein said upper member comprises an upper trough for receiving said maxillary dentition and wherein said lower member comprises a lower trough for receiving said mandibular dentition, wherein at least some of said adjustment bar extends anteriorly of said lower trough.

3. The dental appliance as defined in claim 2, wherein said first element extends anteriorly of said lower trough.

4. The dental appliance as defined in claim 2, wherein most of said adjustment positions are located anteriorly of said lower trough.

5. The dental appliance as defined in claim 2, wherein at least part of said adjustment bar extends above a lower lip of said patient when said appliance is worn by said patient.

6. The dental appliance as defined in claim 1, wherein a posterior wall of said appliance extends less than 5 mm posteriorly of said lower trough.

7. The dental appliance as defined in claim 1, wherein said first element comprises:
(a) a first plate mounted on said upper member; and
(b) a second plate releasably connectable to said first plate, wherein said first and second elements together define said cavity.

8. The dental appliance as defined in claim 1, wherein said connector comprises a head formed at said first end of said connector and a shaft extending downwardly from said head, wherein said second end of said connector is an end portion of said shaft remote from said head.

9. The dental appliance as defined in claim 8, wherein said adjustment positions are threaded apertures and wherein said second end of said connector is threadedly connectable to a selected one of said threaded apertures.

10. The dental appliance as defined in claim 4, wherein at least some of said adjustment positions extend anteriorly of said upper trough.

11. The dental appliance as defined in claim 1, wherein said second element is T-shaped.

12. A connecting assembly for use in a dental appliance for wear by a patient having class II malocclusion, said appliance having an upper member conforming to the patient's maxillary dentition and a lower member conforming to the patient's mandibular dentition, said connecting assembly comprising:
(a) a first element mountable on said upper member, said first element having a cavity therein;
(b) a second element mountable on said lower member, said second element comprising a posterior portion and an adjustment bar extending anteriorly from said posterior portion and having a plurality of spaced-apart adjustment positions formed therein; and
(c) a connector having a first end captured within said cavity and a second end adjustably connectable to said second element,
wherein said connecting assembly maintains said patient's mandible in an anteriorly protruded position relative to its normal, unadjusted position while permitting a limited degree of movement of said lower member relative to said upper member in said anteriorly protruded position.

13. The connecting assembly as defined in claim 12, wherein said assembly comprises:
(a) a first plate mountable on said upper member; and
(b) a second plate releasably connectable to said first plate, wherein said first and second elements together define said cavity.

14. The connecting assembly as defined in claim 13, wherein said connector comprises a head formed at said first end of said connector and a shaft extending downwardly from said head, wherein said second end of said connector is an end portion of said shaft remote from said head.

15. The connecting assembly as defined in claim 14, wherein said second end of said connector is threadedly connectable to a selected one of said apertures of said adjustment bar.

16. The connecting assembly as defined in claim 12, wherein said adjustment positions are threaded apertures and wherein said second end of said connector is threadedly connectable to a selected one of said threaded apertures.

17. The connecting assembly as defined in claim 12, wherein said second element is T-shaped.

18. A method of treating a sleep disorder in a patient having class II malocclusion by adjustably maintaining the patient's mandible in an anteriorly protruded position relative to its normal, unadjusted position, comprising:
(a) casting an upper member by forming an upper trough therein conforming to the patient's maxillary dentition;
(b) casting a lower member by forming a lower trough therein conforming to the patient's mandibular dentition;
(c) securing a first connecting element to an undersurface of said upper member, said first connecting element comprising a cavity and a connector having an upper end loosely captured in said cavity, a downwardly extending shaft and a lower end on said shaft remote from said upper end;
(d) securing a second connecting element to an upper surface of said lower member, said second connecting element comprising a posterior portion and an adjustment bar extending substantially anteriorly of said lower trough, said adjustment bar having a plurality of adjustment positions;
(e) determining a preferred protruded position of said mandible required to alleviate the patient's sleep disorder; and
(f) releasably securing said lower end of said connector to a selected one of said adjustment positions corresponding to said preferred protruded position as determined in step (e), wherein said connecting element permits a limited degree of excursion of said mandible while maintaining said mandible in said preferred protruded position.

19. The method as defined in claim 18, wherein a substantial portion of said second connecting element is secured to said lower member anteriorly of said lower trough so as not to substantially constrain the patient's tongue in the protruded position.

20. The method as defined in claim 18, wherein said sleep disorder is selected from the group consisting of snoring, sleep apnea and nocturnal bruxism.
